(19) United States
(12) Patent Application Publication
(10) Pub. No.: US 2010/0087755 A1
(43) Pub. Date: Apr. 8, 2010

(54) MEDICAL/SURGICAL TOOL FOR BOTH IDENTIFYING SUBCUTANEOUS TARGET TISSUE BY MEANS OF EMITTING AN INTERROGATION SIGNAL AND POSITIONING A CATHETER AT THE TARGET

(76) Inventor: Andre P. Boezaart, Gainesville, FL (US)

Correspondence Address: INTEL. PROP./ RND;STRYKER CORPORATION, 4100 EAST MILHAM AVE., KALMAZOO, MI, 49001-6197, US

(21) Appl. No.: 12/571,478

(22) Filed: Oct. 1, 2009

Related U.S. Application Data
(63) Continuation of application No. PCT/US2008/059004, filed on Apr. 1, 2008.
(60) Provisional application No. 60/909,502, filed on Apr. 2, 2007.

(51) Int. Cl. A61M 25/00

(52) U.S. Cl. 600/585

(57) ABSTRACT

A tool (10) for introducing a catheter or wire into subcutaneous tissue including a housing (11). A needle extends forward from the housing so as to serve as the member through which the catheter or wire is introduced. An introducer assembly (40, 240) attached to the housing allows the practitioner to, with the hand the holds the housing advance/retract the catheter or wire. Other control members (62a, 62b) on the housing allow the practitioner to, with the same hand, regulate the level of the stimulating signal sourced to the needle, catheter or wire and to select to which component the needle or the conductor associated with the catheter or wire, to which the stimulating signal is applied.

### RELATIONSHIPS TO EARLIER FILED APPLICATIONS

This application is a continuation of PCT App. No. PCT/US2008/059004 filed 1 Apr. 2008. PCT App. No. PCT/US2008/059004 is a non-provisional of U.S. Provisional Pat. App. No. 60/909,502 filed 2 Apr. 2007. The contents of the above-identified applications are explicitly incorporated herein by reference.

### FIELD OF THE INVENTION

The present invention is directed toward a tool that integrates a stimulating needle and a catheter insertion assembly into a single handpiece. More particularly, the tool of this invention can be both held and positioned by a medical practitioner with one hand.

### BACKGROUND OF THE INVENTION

There are a number of different medical procedures the goal of which is to deliver a therapeutic agent to a specific location within the body of the patient. One such procedure in which it desirable to so deliver an agent, is known as a nerve block procedure. A “nerve block” procedure is performed to lessen pain emanating from a particular region of the body. In this procedure, an anesthetic is introduced to the nerve downstream from the location where the pain is emanating. The anesthetic is introduced through an elongated flexible tube known as a catheter. The anesthetic blocks the down line transmission of the pain signals by the neural network to the brain. By selectively introducing anesthetic to the nerve from which the pain signals are being transmitted, the need to use another pharmaceutical that may have effects throughout the body is eliminated. The elimination of these body wide pharmaceuticals results in the like elimination of the side effects associated with their use.

An important step in performing a nerve block or other procedure in which the goal is to precisely introduce a therapeutic agent at a target site is the precise positioning of the distal open end of the catheter at the site. When the procedure is a nerve block, the distal end of the catheter should be positioned in close proximity to the nerve through which the pain signal is transmitted. This process must be performed carefully because nerves and surrounding tissue are fragile structures. This necessitates the need for a medical device that can precisely locate a nerve without damaging it. In one known method of nerve location, using known geographical locations on the patient, i.e. leg, arm, bone structure, etc., a medical practitioner inserts a needle having a central bore, into the patient close to the nerve to which the therapeutic agent (anesthetic) is to be applied. While inserting the needle with one hand, the practitioner usually uses his/her other hand to operate a separate device, such as an ultrasound device. The ultrasound device produces an image of the underlying tissue. The image created by the ultrasound device serves as a further guide so that the medical practitioner not only inserts the needle relatively close to the desired nerve, but also avoids passing the needle through other tissues, i.e. tendons and vascular structures, that could be harmed if punctured or torn.

To determine if the needle is in close proximity to the nerve, it is a known practice to emit an electric signal from the needle. As the needle approaches the nerve, the resultant electric field around the needle stimulates the nerve, i.e. creates muscle twitches or other responses. These responses provide the practitioner feedback that the needle is adjacent the nerve.

Once the needle is properly placed, a catheter is inserted into the bore of the needle. It is crucial that the insertion of the catheter into the needle does not alter the placement of the needle. Presently, to minimize needle displacement, the practitioner often lets go of the ultrasonic probe and uses the hand now freed to insert and advance the catheter through the needle towards the subcutaneous target site. While performing this task, the practitioner must still hold the needle as steady as possible so as not to disrupt its original placement.

To ensure proper positioning of the catheter, often a signal is applied to some sort of electrode adjacent its distal end opening. Like the needle, the electric field caused by the electrical signal supplied to the catheter probe stimulates the nerve. This provides the practitioner information regarding the final placement of the catheter. Upon correct placement of the catheter, the therapeutic agent (anesthetic) is introduced through the catheter to the desired nerve of the patient. The needle is typically pulled out. This leaves only the fluid delivery catheter inserted in the patient.

Difficulties arise in the above process when the medical practitioner holds the needle in one hand while advancing the catheter with the other hand. With both hands in use, the medical practitioner is unable to operate the ultrasound probe. Without ultrasound, the medical practitioner is unable to visually track and guide the progress of the catheter within the patient and must solely rely upon the response caused by the electrical signal of the catheter to provide feedback whether the catheter has been correctly placed. This increases the risk of needlessly tearing or puncturing tissues, i.e. arteries, within the patient.

To overcome the above problem, a practitioner will sometimes rely on an assistant to position the ultrasound probe. This requires the practitioner and the assistant coordinate simultaneous needle/catheter insertion and ultrasound probe placement. As when any two individuals are required to coordinate simultaneous activity, this adds to the complexity of the procedure.

### SUMMARY OF THE INVENTION

This invention relates to a new and useful medical/surgical catheter insertion tool that allows a medical practitioner to accurately locate a particular anatomical site of a patient, such as a nerve, and position a catheter at the site. More specifically, this medical/surgical catheter insertion tool includes a housing from which a needle, such as stimulating needle extends. Integral with the housing is a catheter insertion assembly. The housing also contains actuators for both controlling the level and trigging of the application of the signal applied to the needle or catheter. The housing is shaped in such a way that allows the medical practitioner to hold the tool with one hand.

The needle of the tool of this invention extends from the housing and has a central bore. The needle may be coated with an insulating material, except at its end most distal from the housing. An electrical signal is supplied to the needle. This signal forms an electric field around the distal end tip of the needle.

The catheter introducer assembly is disposed inside the housing. This assembly allows the practitioner to move the catheter through and out of the distal end of the needle. If the catheter is of a type to which a stimulating signal can be applied, the housing also includes the regulators for controlling the output of this signal from the catheter.

In some alternative versions of the invention, an alternative introducer assembly is disposed in the housing. This introducer assembly allows the practitioner to selectively advance/retract a wire such as a guide wire. Once the distal end of the guide wire is positioned at the target site, a catheter can be fitted over the guide wire. This results in the proper positioning of the catheter. Once the catheter is so positioned, the guide wire is withdrawn from the catheter. The practitioner is then able to introduce the therapeutic agent into the catheter so that it will flow to and be discharged at the desired target site.

It should be appreciated that in some procedures, the catheter can be implanted so as to serve as a drainage device.

### BRIEF DESCRIPTION OF THE DRAWINGS

The invention is pointed out with particularity in the claims. The above and further features and benefits of this invention are understood by reference to the following Detailed Description taken in conjunction with the accompanying drawings in which:

FIG. 1A is a side view of the first embodiment of the medical/surgical insertion tool of this invention;

FIG. 1B is an isometric view of the first embodiment of the medical/surgical insertion tool;

FIG. 1C illustrates an alternative catheter advancement/retraction system integrated with the medical/surgical insertion tool;

FIG. 1D is a diagrammatic view of how stimulating signal is applied to the needle of the tool of this invention or the catheter that is positioned using the tool;

FIG. 1E is a side view of an alternative tool of this invention illustrating an alternative catheter/wire advancement system of this invention;

FIG. 2 is a side view of a monopolar catheter embodiment;

FIG. 3 is a cross sectional view of the catheter of FIG. 2;

FIG. 4 is a side view of a bipolar catheter embodiment;

FIG. 5 is a cross sectional view of the catheter of FIG. 4; and

FIG. 6 is a side view of a further embodiment of the present invention

### DETAILED DESCRIPTION

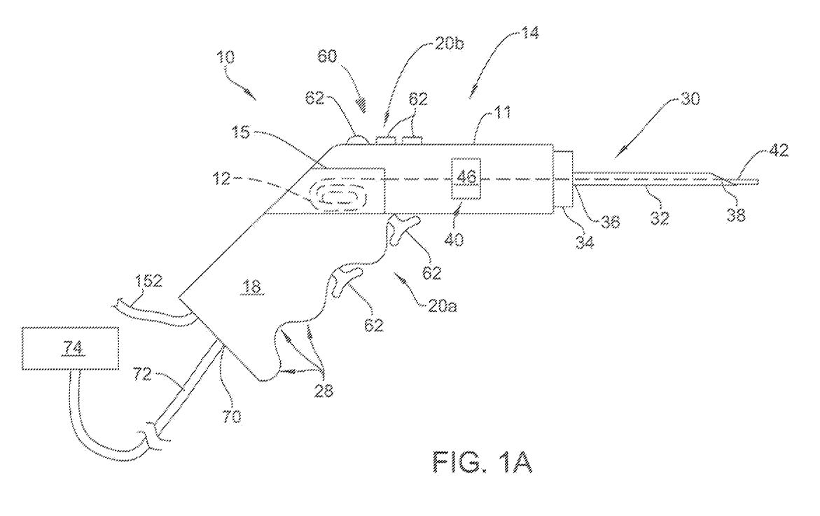

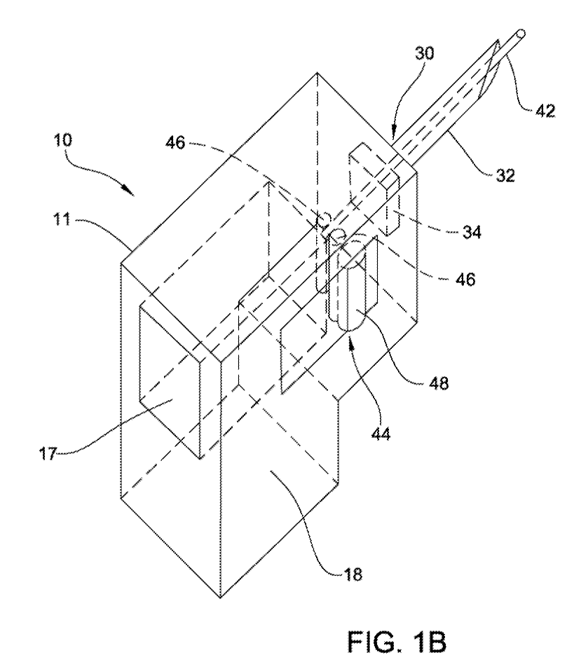

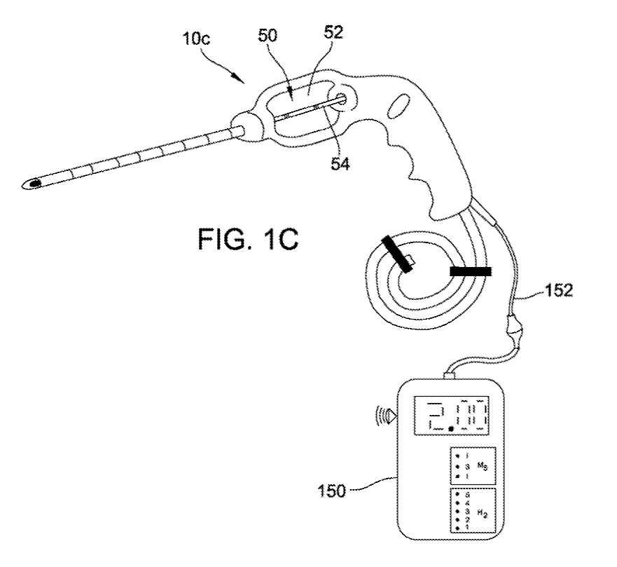

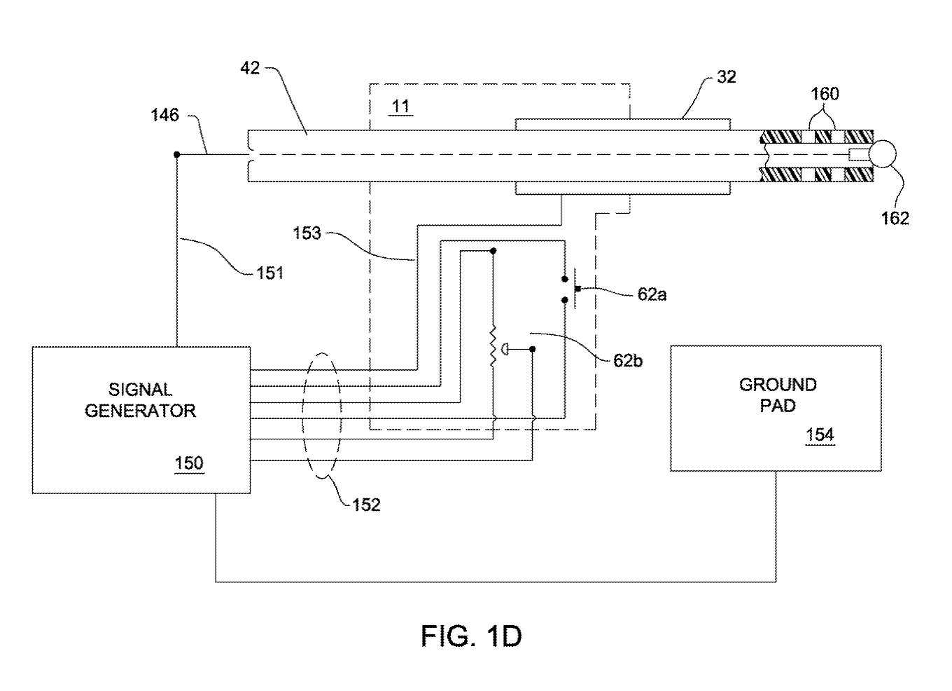

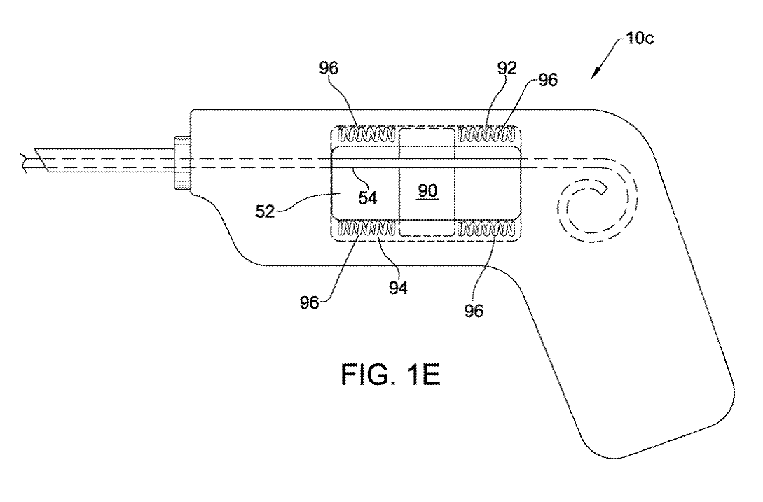

A first embodiment of the medical/surgical insertion tool 10 is shown in FIG. 1A. Since tool 10 is used to insert a catheter 42 it is sometimes referred to below as a catheter inserter. As discussed below, other tools of this invention may be used to insert medical devices other than catheters. Insertion tool 10 comprises three sub-assemblies. A needle assembly 30 includes a needle 32 through which the catheter is introduced to the subcutaneous regions of the patient. Needle assembly 30 also provides a stimulating current. A catheter introducer assembly 40 positions a catheter at the target anatomical site. This is so the therapeutic agent (fluid) discharged from the catheter will have the desired therapeutic effect. A control assembly 60 regulates the application of the stimulating current and the advancement/retraction of the catheter 42. Needle assembly 30, catheter introducer assembly 40, and the primary actuatable controls of control assembly 60 are mounted to a single housing 11. Housing 11 is shaped to allow one handed use of the tool 10.

Needle 32 of needle assembly 30 is attached to a hub 34 attached at the proximal end 36 of the needle. (“Proximal” it should be understood means towards the practitioner performing the procedure. “Distal” means away from the practitioner, towards the target site internal to the patient.) The needle 32 is hollow, having a central bore (not identified) that extends axially through the needle. Needle 32 may either be formed from conductive or non-conductive material. When formed from conductive material, the needle 32 is typically at least partially coated or surrounded with an insulating material; the proximal end 36 and distal end 38 usually being the exceptions. When formed from non-conductive material, the needle is often formed to have a conductive ring around the distal end tip. This conductive ring may be formed from a metal coating.

To provide the medical practitioner a means to find a desired anatomical target site internal to the patient, i.e., a nerve, an electrical signal is typically supplied to the needle 32. This signal can be considered an interrogation signal. FIG. 1D illustrates the basic assembly for applying this signal. Specifically, the signal originates from a signal generator 150. The signal generator 150 outputs a DC signal. Often the current of the DC signal can be adjusted. Typically, the current is between 0.1 mA and 1.5 mA. If the signal generator 150 outputs an AC signal, the frequency is typically adjustable. If the signal generator is able to output a DC signal, the signal may be of fixed pulse length and/or a pulse wave signal. As described below, the signal generator is remotely configurable and actuatable. Thus, based on remotely generated signals, the signal generator 150 both establishes the characteristics of the stimulating signal and selectively applies the signal to either the needle 32 or the distal end of the catheter 42. The actual structure of the signal generator 150 is not the subject of this invention.

The stimulating signal produced by signal generator is supplied to the tool housing 11 over cable 152. In FIG. 1D, conductor 153 is the conductor over which the signal is supplied to the tool housing 11. Internal to housing 11, the signal is applied to the needle 32 over a conductor 153. In some versions of the invention the conductor 153 is solder secured or otherwise fixedly secured to the needle 32. Another alternative means of so connecting the conductor 153 is with a screw. Alternatively, a component internal to the housing may provide a conductive path from conductor 153 to the needle without actually being fixedly connected to the needle. A conductive spring such as a leaf spring or a coil spring may perform this function.

As discussed below, both the setting of the characteristics of the stimulating signal and the application of the signal is regulated by control assembly 60.

When inserted into the patient and a current is applied thereto, the electrified needle 32 stimulates the nerve(s) with the electric field produced around the uninsulated portions of the needle in contact with the tissue of the patient, i.e. the distal end 38. Needle 32 may comprise a monopolar needle. It is well known in the art that use of a monopolar needle requires a supplemental conductive material attached to the patient, commonly referred to as a ground pad 154, shown diagrammatically in FIG. 1D. When such a needle 32 is employed, the ground pad is usually attached to the patient by means of a sticky adhesion. The ground pad 154 serves as the electrode over which the return signal is applied back to the signal generator 150. The ground pad is not part of this invention. It is to be appreciated that a bipolar needle may also be used in conjunction with the tool 10. A typical bipolar needle has two conductive surfaces exposed, one is connected to ground and the other is supplied with the electrical signal. In this version of the invention, cable 152 also includes the conductor over which the return signal is forwarded back to the signal generator 150. (Return conductor not illustrated.) While not shown, it should be appreciated that conductor 151, the conductor over which a signal is applied to catheter conductor 164, may be contained in cable 152.

The hub 34 is coupled to the proximal end 36 of the needle 32. Hub 34 is formed with features that allow the needle assembly 30 to be coupled to the housing 11. This is to accommodate medical practitioners who wish to initially insert the needle 32 without having to hold housing 11. Thus, the types of coupling features with which the hub can be provided include threading, a latch, snap clips or a magnet. These features also allow the practitioner to decouple the needle assembly 32 from the housing 11.

The hub 34 also provides means to axially align the central bore of the needle 32 with the catheter introducer assembly 40. This facilitates the alignment facilitates the movement of the catheter 42 from the housing 11 into the needle 32.

Catheter introducer assembly 40 positions the catheter 42 in the patient so that the distal end of the catheter, the end that projects forward of needle 32, is located at or adjacent the tissue (target site) to which the pharmaceutical agent is to be applied. Catheter introducer assembly 40 also includes components capable of applying a stimulating signal to the tissue adjacent the distal end of the catheter 42. This stimulation can be used as a guide for positioning the distal end of the catheter.

Catheter 42 is formed from non-conductive material. The catheter 42 has an open distal end. As shown in FIG. 1D, the catheter 42 is formed to have fenestration openings 160 that extend radially through the catheter so as to function as through ports from the central lumen of the catheter. Fenestration openings 160 are immediately proximal, rearward, of the distal end. Openings 160 are the openings through which the therapeutic (pharmacological) agent is flowed from the catheter to the surrounding tissue. A small ball 162 formed of a conductive metal is disposed in the distal end opening of the catheter 42. Ball 162 is the conductive member to which the stimulating signal is applied to determine the location of the distal end of the catheter 42. A conductor 164, shown partially as a dashed line, extends from outside the proximal end of the catheter, through the catheter lumen, and is connected to ball 164. The proximal end of conductor 164 is connected to signal generator 150 by a conductor 151. Conductor 164 serves as the conductive member over which the stimulating signal is applied to ball 162.

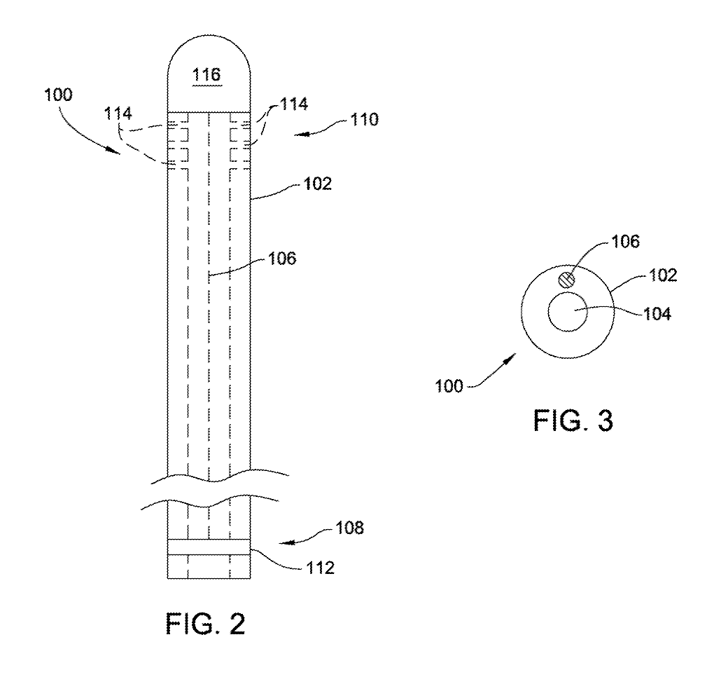

An alternative catheter 100 is shown in FIGS. 2 and 3. Catheter 100 comprises a tubular structure 102 with a lumen 104 that extends axially through the length of the tubular structure. A wire 106 is disposed within the tubular structure 102; extending from the proximal end 108 to the distal end 110. It is to be appreciated that the wire 106 may also be disposed in the lumen 104. At the proximal end 108, the wire 106 connects to a conductive band 112 disposed on the outside of the catheter 100. Conductive band 112 serves as the exposed conductive member of the catheter to which the cable from a signal generator 150 is connected. Distally forward of the conductive band 112, the wire 106 travels to the distal end 110 in a linear fashion. However, it is to be appreciated that a helical wire could also be used as wire 106 or a combination of helical and straight wire. At the distal end 110 of the catheter 100, the wire 106 is attached to the conductive tip 116. When a stimulating signal is applied to the conductive tip 116, an electric field develops around the tip. Also at the distal end of the catheter 100, the tubular structure 102 has at least one fenestration opening 114 to permit fluid delivered through the lumen 104 to exit the catheter 100 around the distal end 110

Catheters 42 and 100 are “monopolar” catheters. Thus, one is required to provide ground pad 154 as the return electrode for the signal output from the catheter active tip (electrode).

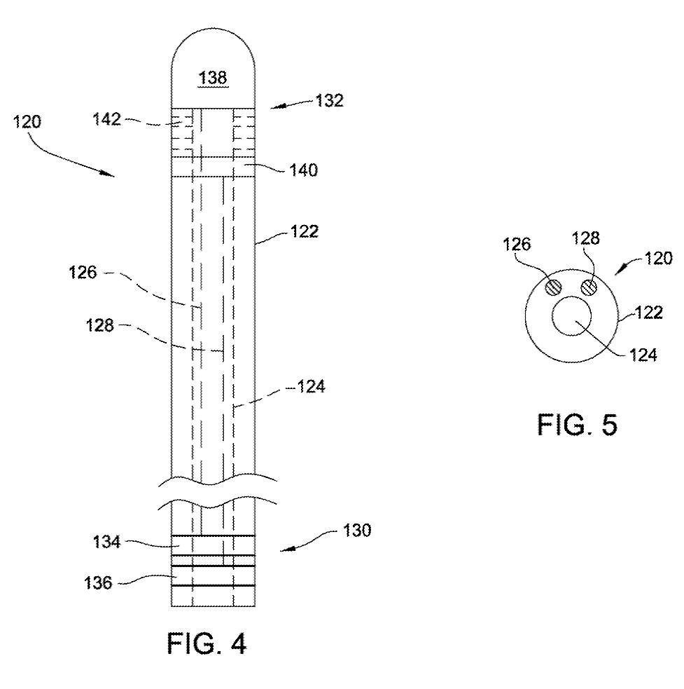

Referring to FIGS. 4 and 5, an example of a bipolar catheter 120 is shown. Catheter 120 is a tubular structure 122 with a lumen 124 that extends axially through the tubular structure. A first wire 126 and a second wire 128 are disposed within tubular structure 122, extending from the proximal end 130 to the distal end 132. Though illustrated as being disposed within the structure 122, the first and second wires 126 and 128, respectively, could also be disposed in the lumen 124. At the proximal end, two conductive bands are wrapped around the tubular structure 122; a first conductive band 134 and a second conductive band 136. First wire 126 connects to the first conductive band 134. Distally forward of the first conductive band 134, the first wire 126 is disposed in a linear fashion in the outer wall 123. It is to be appreciated that the first wire 126 could also be helical or a combination of helical and straight. At the distal end 132, the first wire 126 connects to the conductive tip 138 which is distally forward of the tubular structure 122. Second wire 128 is connected to a second conductive band 136 at the proximal end. Like the first wire 126, the second wire 128 may also comprise either of a linear or helical wire. At the catheter distal end 132, the second wire 128 connects to a third conductive band 140 that is wrapped around the tubular structure 122. At least one fenestration opening 142 is formed in the catheter distal end 132.

First conductive band 134 may be connected to the conductor internal to the housing 11 over which the active signal is applied from the signal generator 150. Second conductive band can be connected to the conductor internal to the housing that is connected to the return terminal of the signal generator 150. Third conductive band 140 acts as the return electrode on the catheter 120. The electric signal supplied to the conductive tip 138 grounds to the third conductive band 140. This produces an electric field around the distal end 132 of the catheter 120. This field can stimulate nerves adjacent tip 138 and band 140.

Introducer assembly 40 for advancing and retracting the catheter 42 in and out of the patient, through the central bore of the needle 32, is now described. It is preferable that the means provided to advance/retract the catheter 42 provides the medical practitioner with tactile feel of the catheter advancement/retraction. The practitioner uses this tactile feel and relates it to known information, i.e. what kind of tissue the catheter 42 is encountering. The practitioner also uses this tactile feedback to determine if the catheter has encountered an object in the patient's body that blocks further advancement.

In the disclosed version of the invention, seen in FIG. 1B, a frictional engaging system 44, part of the introducer assembly 40, engages the catheter 42 to advance/retract the catheter. The frictional engaging system 44 may include any number and any type of catheter displacement rollers 46 that frictionally engage the catheter 42. In the illustrated version of the invention, rollers 46 are parallel cylinders that are rotatingly mounted to the tool housing 11. Catheter 42 is compressed between rollers 46. The rollers 46 can be formed from resilient sterilizable material. Rotation of rollers 46 causes translational movement of the catheter 42 sandwiched therebetween. In other versions of the invention the frictional engaging members that actually displace the catheter may comprise of wheels, translational sliders, polygonal structures, rotational members, translational members, ratchet-type members, guide elements, etc. Each frictional engaging member may be used independently or in conjunction with any other type of frictional engaging member to create the frictional engaging system 44.

Catheter introducer assembly 40 may also transducers capable of either directly or indirectly determining the extent to which the catheter 42 is advanced or retracted. The signals output by these transducers may be applied to the control assembly 60. Control assembly 60 can use these signals as feedback signals to regulate the actuation of the catheter introducer assembly. In addition to measuring catheter advancement/retraction, one or more transducers may also be used to determine the velocity or acceleration of the catheter 42. Again, data representative of catheter velocity may be used as feedback signals for regulating the components of the catheter introducer assembly 40 that advance/retract the catheter.

In the illustrated introducer assembly 40, catheter introducer assembly also includes a cylindrical drive roller 48 for actuating catheter displacement rollers 46. Drive roller 48 is rotatably mounted to the housing 11 for fingertip actuation. Driver roller 48 extends partially through a window formed in the housing sidewall (window not identified). Drive roller 48 abuts and actuates one of the catheter displacement rollers 46. Drive roller 48 thus functions as the manually actuated member that drives the roller 46.

In some versions of the invention, the cylindrical wheels rotation is monitored by a transducer. The electrical signal output by the transducer is employed to generate data regarding catheter advancement/retraction to the control assembly 60. To operate the roller 48, the medical practitioner typically uses his/her finger(s) to roll the wheel in either direction. Rotation of roller 48 in a first direction advances the catheter 42 into the patient; rotation in the opposite direction of rotation retracts the catheter out of the patient.

Another method of advancing/retracting catheter comprises system 50 which seen in FIG. 1C. The medical/surgical catheter insertion tool 10c typically has an space 52 that allows finger (preferably thumb and index) access to the catheter 54 for the medical practitioner. This space allows the medical practitioner's fingers to operatively engage the catheter with simple push/pull techniques to advance/retract the catheter. As seen in FIG. 1E, tool 10c can include a slide plate 90 that is mounted to the barrel of housing 11c, the portion of the housing that defines space 52. The top and bottom ends of plate 90 are seated in elongated slots 92 and 94, respectively, formed in the housing. A pair of springs 96 is also mounted in each slot 92 and 94. In each slot 92 and 94, the slide plate 90 is disposed between the springs 96. Springs 96 normally hold the slide plate 90 in a centered position within space 52. It should further be understood that the slide plate 90 is positioned to be adjacent catheter 54. In some versions of the invention, the slide plate 90 is formed with a laterally extending groove in which the catheter 54 is seated.

Catheter 54 is advanced by the practitioner pressing his/her finger against the catheter and the adjacent slide plate 90. The catheter 54 is thus captured between the finger and the slide plate 90. The practitioner then advances/retracts the finger to cause a like displacement of the catheter. As the practitioner finger reaches the end of the advancement/retraction finger cycle, the practitioner releases the finger from the catheter 54 and slide plate 90. The release of the finger pressure causes the springs 96 to return the slide to the plate 90 to its static position while the catheter remains in its advanced/retracted position. The practitioner can then place the finger back against the catheter and slide plate 90 to further adjust the position of the catheter 54.

An advantage to the system 50 is that it allows the medical practitioner to view if a body fluid has entered the catheter.

Control assembly 60 allows the medical practitioner to regulate a number of processes that can be performed with tool 10 of this invention. The control assembly 60 includes at least one control member 48 or 62, i.e. pushbuttons, trigger mechanisms, wheels, switches, levers, rotational members, translational members, hinge members, etc. Each control member 48 or 62 may control one or plural functions of the tool 10. Drive roller 48 of the version of the invention described with respect to FIG. 1B thus functions as the control member used to actuate the catheter introducer assembly 40.

For instance, engagement of control member 62a, such as a trigger or pushbutton, is used to selectively assert a first digital signal to signal generator 150. When the signal generator 150 is in a first state, the depression of control member 62a results in an assertion of a signal to the signal generator 150 that causes the signal generator to source the stimulating signal over conductor 153 to the needle 32. When the signal generator 150 is set to a second state, established by a second setting of control member 62a, the output signal from the signal generator 150 is sourced to the catheter conductor 164 and by extension the catheter conductive tip 162. Control member 62a should be understood as potentially having a third state in which the member is placed when there is no need to source a stimulating signal to either the needle 32 or the catheter 42.

The tool of FIG. 1D is further shown as having a control member 62b capable of output a variable level DC signal. In the Figure, the control member 62b is represented by the potentiometer. Signal generator 150 is configured to, based on the voltage level of the signal regulated by control member 62b, set the current level of the stimulating signal output to the needle or catheter tip. By adjusting control member 62b the voltage of the stimulating signal sourced to the needle 32 or catheter 42 or 54 can be set between a zero volts off state, a maximum voltage or a voltage level therebetween.

Some versions of the invention may have three or more control members. In versions of the invention, catheter introducer assembly 40 includes a motor used to advance/retract the catheter. In these versions of the invention an additional control member, member 62c of FIG. 1A, is provided. This third control member 62c is selectively actuated to regulate the actuation of the motor. Still another housing mounted control member may be used to control the functionality of another control member. Thus, a first control member may be used to control to which of the electrodes, the needle or the catheter, the signal generator should output the stimulating signal. A second control member, as described above, is used to actually cause the signal generator to output the stimulating signal.

As can be seen in FIG. 1A, the needle assembly 30, catheter introducer assembly 40, and the control assembly 60 are each built into or attached to housing 11. The integration of each assembly 30, 40, and 60 enables the medical practitioner to effectively operate the medical/surgical catheter insertion tool 10 and its associated features with one hand.

In the drawings, housing 11 is shown generally as being pistol shaped; there is barrel 14 from which the needle and catheter extend and grip 18 below the barrel. Tool 10 of this invention is not limited to housing having this particular shape. For example, in alternative tool of this invention may have a housing with an elongated pencil-like shape. Alternatively, the housing may have a proximal butt end that is angled slightly downwardly from the distal front end to form a hand grip. The housing 11 preferably consists of a plastic or metal.

In the illustrated version of the invention, barrel 14 is the component from which needle assembly 30 extends. Introducer assembly 40 and control assembly 60 includes control members located on the grip 18 and barrel 14.

The grip 18 extends proximally angularly away from the barrel 14. The angle relative to the grip 18 and the barrel 14 is preferably between 90° and 180°, more preferably between 110° and 160°, and most preferably between 120° and 140°. In the first embodiment, the grip 18 may have finger groove features 28 that face the distal portion of the medical/surgical catheter insertion tool 10. The groove finger features 28 provide the medical practitioner an ergonomic feel when holding the tool 10.

The grip 18 may also include an at least one user control section to provide the medical practitioner means for operating the tool 10, such as, the advancement and retraction of the catheter 42, modify the electrical signal supplied to the needle 32 and catheter, control the engagement of the needle assembly 30 with the tool 10, and deliver fluid to the patient. As shown in FIG. 1A, two user control sections are illustrated; a first user control section 20a and a second user control section 20b. Each user control section 20a, 20b may comprise of one or more control members 62. The first control section 20a is disposed on the distal face of grip 18. The location of the first user control section 20a allows the medical practitioner to operatively engage features of the tool 10 with his/her fingers, while holding the device with one hand. For instance, first control section 20a may be used in conjunction with the frictional engaging system 44. In other words, the control members 62 of the first control section 20a may advance/retract the catheter by means of integration with the friction engaging system 44. The second user control section 20b is located on the forward facing surface of the grip 18. Typical features regulated by the controls of section 20b include the on/off state of the stimulating signal and to which component, the needle 32 or the catheter tip 162 the signal is applied.

Grip 18 may further include a void space 17 (FIG. 1B) for receiving at least one insertable/removable cartridge 15. As shown in FIG. 1A, cartridge 15 houses the catheter 42 up line of the catheter introducer assembly 40. Upon inserting the cartridge 15 into the medical/surgical catheter insertion tool 10, a sensor reads whether the cartridge is seated correctly in the tool. Once the cartridge 15 is inserted or seated correctly in the tool 10, the sensor sends an input signal to the control assembly 60. The control assembly 60 may alert the user via a speaker or LED array that the catheter 42 is ready for manual advancement so it is engaged by the catheter introducer assembly 40. Though described as a catheter storage device, the cartridge 15 may also or instead contain a rechargeable battery to supply at least some power to the tool 10. The battery could provide power to the control assembly 60, allowing for self-powered inputs and outputs that do not rely on power from a connected supplemental device.

A supplemental unit 74 may be connected to tool housing 11 by a cable 72. Supplemental device 74 may operate as a power supply, an irrigation device, a nerve stimulation device, etc. The cable 72 typically has means to provide necessary functionality between the supplemental device 74 and the tool 10.

It should be appreciated that the tool 10 can either be made of sterilizable material or disposable material. If made of sterilizable material, the tool 10 may include a catheter storing cartridge 15 as being disposable. By making the tool 10 out of plastic, thus disposable, the tool's weight can be significantly lighter than that of a device made to stand the process of sterilization.

Use of the medical/surgical catheter insertion tool 10 is as follows: The medical practitioner, operatively holding the medical/surgical catheter insertion tool 10 in one hand, inserts the needle 32 into the patient. Because the medical/surgical catheter insertion tool 10 is held in one hand, the medical practitioner can hold a supplemental device, such as an ultra sound probe, in their other hand. The ultra sound probe aids the practitioner in needle 32 placement.

Using the image produced by the ultrasound device as a visual aid, the practitioner advances the needle 32. During the needle advancement process, the practitioner, by actuating one of the control members 62, selectively applies a signal to the distal end conductive component of the needle 32. When the needle is the vicinity of a nerve, the signal output by the needle stimulates the nerve. The practitioner monitors the patient to determine if he/she undergoes a physiological response to such stimulation, (often involuntary muscle movement.) Thus this stimulating signal can be considered an interrogation signal. During this process, the practitioner may vary the parameters of the electrical signal supplied to the needle 32 with another one of the control features 62. Ultrasound imaging coupled with variable electrical signal stimulation allows the medical practitioner to achieve desired needle placement.

Once the needle 32 is in the general vicinity of the desired nerve, the practitioner adjusts the electrical parameters of the stimulating signal supplied to the needle 32, such as voltage or current. By lowering the voltage or current of the output signal the practitioner can more accurately determine the position of the distal end of the needle 32 relative to the nerve.

Once the needle 32 is correctly placed, the medical practitioner advances the catheter 42 into the patient using the introducer assembly 40. Since the catheter introducer assembly 40 is contained within housing 11, the practitioner, with a single hand, is able to both position and advance the catheter.

During the insertion of the catheter 42 or 54, the practitioner, by actuation of the appropriate control feature, selectively causes a signal to be output from catheter tip 162. Again, the practitioner monitors the patient for physiological responses that indicate the distal end of the catheter is adjacent the targeted tissue. This aids the medical practitioner in facilitating the proper positioning of the catheter 42. Once the catheter 42 is correctly placed, catheter conductor 164 is disconnected from the signal generator. Tool 10 is retracted away from the patient by sliding the needle 32 over the catheter. Once the tool is removed, the catheter is fixated to the patient and coupled to a container holding the pharmaceutical agent. The agent is then delivered through the catheter to the desired anatomical site.

### Alternative Embodiments

The foregoing describes only certain embodiments of the insertion tool of this invention. Alternative embodiments of this tool are possible.

For example, in alternative version of this invention, the tool of this invention is not limited to tools capable of precision positioning a catheter into a patient. One alternative tool of this invention includes an insertion assembly capable of introducing a wire into a patient. In some versions of the invention, the placement of the wire serves as the final placement of the medical device in the patient. These wires are thus wires that contain electrodes or transducers designed to perform a therapeutic effect or that are used to monitor subcutaneous tissue that cannot otherwise be easily monitored.

Alternatively, the wire functions as a guide member over which a catheter is introduced to a target site internal to the patient. In this type of procedure, the wire is referred to as a guide wire. In this type of procedure, once the guide wire is properly positioned, a catheter is slip fitted over the guide wire. Since the guide wire terminates at the desired target location internal to the patent, the catheter can be feed over the guide wire to reach this location. Once the catheter is properly positioned, the guide wire is withdrawn from the catheter. Therapeutic agent can then be introduced to the target site through the catheter. Alternatively, the catheter may be inserted to function as a drainage tube.

Again, other devices such as ultrasound monitoring can be used to determine the position of the guide wire.

It should likewise be clear that tool of this invention can be used to position the medical device at target locations in the body other than nerves. Thus, the tool can be used to provide the medical practitioner access to a particular anatomical site, e.g. hollow organ, cardiovascular vessel, i.e. artery or vein, joint or cavity. When a guide wire is used to so position a catheter in a hollow organ or blood vessel, this procedure is sometimes referred to as the Seldinger technique.

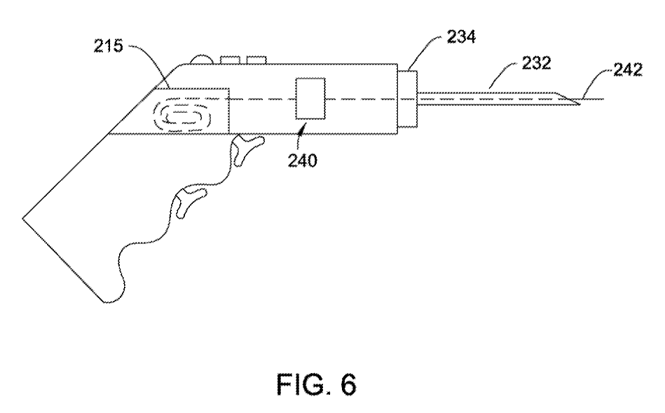

As illustrated in FIG. 6, medical/surgical guide wire insertion tool 200 comprises like features of tool 10. A few differences exist between tool 10 and tool 200; namely, the catheter is replaced by a guide wire 242, the catheter introducer assembly 40 is renamed as the guide wire introducer assembly 240, and cartridge 215 houses the guide wire 242 instead of a catheter.

Use of the medical/surgical guide wire insertion tool 200 is as follows: The medical practitioner, operatively holding the tool 200 in one hand, inserts the needle 232 into the patient. Because the tool 200 can be held in one hand, the medical practitioner may choose to hold a supplemental device, such as an ultra sound device, in their other hand. The ultra sound device is used to aid the medical practitioner in needle 232 placement and to also avoid structures that would be damaged if punctured or torn by the needle, i.e. cardiovascular structures, tendon structures, etc. Using the image produced by the ultrasound device as a visual aid, the medical practitioner advances the needle 232 to the desired anatomical site.

Once needle 232 is correctly positioned, e.g. punctures the desired anatomical site, the medical practitioner advances the guide wire 242 into the patient using the guide wire introducer assembly 240 so as to selectively feed the guide wire through the needle. Since the guide wire introducer assembly 240 is integral to the tool 200 housing, the medical practitioner with a single hand can both position and advance the guide wire 242, thus allowing the medical practitioner to operate a supplemental device, such as an ultrasound device.

Upon the correct placement of the guide wire 242 within the patient, tool 200 is retracted away from the patient by sliding the needle 232 over the guide wire 242. Once the tool 200 is removed, a catheter, cannula, or tubular structure, e.g. drainage tube, is then passed over the guide wire into the anatomical site at which the distal end of the guide wire is present. The guide wire may then be withdrawn from the patient, leaving only the tubular structure positioned in the patient.

Further, it should be clear that there is no requirement that all versions of the invention include the above described features. In some versions of the invention, tool 10 of this invention may not be designed for use in procedures wherein one does not apply an electrical signal to determine the location of the distal end of the needle, catheter or guide wire. Thus, in these versions of the invention, the need to provide the tool housing with means to apply a signal to the needle, catheter or guide wire, as well control members for regulating and trigging the signal are not necessary. Likewise in some versions of the invention, it may only be necessary to provide a housing with features for applying a stimulating signal to just one of the needle, catheter or guide wire.

Similarly, the introducer assembly for the catheter or guide wire may have features different from what has been described. Thus, the tool of FIG. 1C may have a single roller or wheel. This rotating member is disposed in the window through which the catheter/guide wire extends. This rotating member serves as the component against which the practitioner, with the thumb or finger, presses the catheter/guide wire. The catheter/guide wire is advanced by moving the practitioner's digit against the catheter/guide wire. Since the catheter/guide wire is compressed between the practitioner's digit and the rolling member, it will move in the direction in which it is displaced.

Alternatively, the introducer mechanism may include a pawl and ratchet assembly. In some versions of the invention, the introducer assembly may include two pawl and ratchet assemblies, each actuated by its own control member. A first pawl and ratchet assembly is actuated to extend the catheter/guide wire. The second pawl and ratchet assembly is actuated to retract the catheter/guide wire.

A single trigger pawl and ratchet assembly is also within the scope of this invention. In this embodiment of invention, a single trigger may be used to drive the unit to cause both the extension and retraction of the catheter or guide wire. In some versions of this embodiment of the invention, the movement of the trigger in a first direction from an initial state causes the assembly to extend the catheter or guide wire. The movement of the trigger in the second direction, opposite the first direction, causes the assembly to retract the catheter or guide wire.

In an alternative version of this embodiment of the invention, the pawl and ratchet assembly has a second control member such as a lever. When the lever is in a first position, actuation of the trigger causes the pawl and ratchet assembly to extend the catheter or guide wire. When the lever is in a second position, actuation of the trigger causes the pawl and ratchet assembly to retract the catheter or guide wire.

If the introducer assembly includes a drive motor, the tool may include two control members for actuating the motor; a first member for driving the motor to cause catheter/guide wire advancement and a second member for actuating the motor to cause catheter/guide wire retraction.

Similarly, it should be appreciated that there is no requirement that the catheter or guide wire advanced by tool be housed in a removable cartridge. In some versions of the invention, the housing may be formed with a void space in which the catheter or guide wire is preloaded during tool manufacture. Still in other versions of the invention, the tool housing may not have any void space or cartridge receiving space for receiving the tool or cartridge. In these versions of the invention, the housing is formed with an opening through which the catheter/guide wire is feed into the introducer assembly.

Alternatively, in some versions of the invention, the conductive member through which the stimulating current is applied to the catheter or guide wire is disposed in the tool housing.

It should therefore be clear from the foregoing that the tool of this invention has numerous applications beyond positioning a catheter for a nerve block procedure.

Therefore, it is the object of the appended claims to cover all such variations and modifications as come within the true spirit and scope of this invention.

What is claimed is:

1. A catheter/wire introducer, including:
a housing shaped to be held in a single hand;
a needle that extends forward from said housing, said needle having a bore, said needle shaped to be inserted subcutaneously, the needle bore sized to receive a catheter or a wire;
a conductor connected to the needle or a conductor associated with the catheter or wire, wherein a stimulating signal can be applied to the needle or to the conductor associated with the catheter or wire;
a catheter/wire introducer assembly mounted to the housing for advancing the catheter or wire through the needle bore so that a distal end of the catheter or wire can be selectively positioned forward of said needle, said introducer assembly having a manually actuatable component on said housing allowing for user control of the advancement of the catheter or wire, the manually actuatable component positioned to be actuated by the hand holding the housing; and
a first control member is attached to the housing for regulating the level of the stimulating signal applied to the needle or the conductor associated with the catheter or the wire, said first control member positioned to be actuated by the hand holding the housing.

2. The catheter/wire introducer of claim 1 wherein:
the housing includes both said conductor connected to the needle and said conductor associated with the catheter or wire and the stimulating signal can be applied to either said conductor; and
a second control member is mounted to the housing for regulating to which component, the conductor connected to the needle or the conductor associated with the catheter or wire, the stimulating signal from a signal generator is sourced, said second control member positioned to be actuated by the hand holding the housing.

3. The catheter/wire introducer of claim 2, wherein:
a signal generator is separate from the housing supplies the stimulating signal to said needle or said conductor associated with the catheter or the wire; and
said first control member is connected to said signal generator.

4. The catheter/wire introducer of claim 1, wherein said catheter/wire introducer assembly includes a member for engaging the catheter/wire to advance the catheter/wire forward of the needle or retract the catheter/wire back into the needle.

5. The catheter/wire introducer of claim 1 wherein said housing is formed with a window through which the catheter/wire introducer assembly manually actuatable component is accessed.

6. The catheter/wire introducer of claim 1, wherein said catheter/wire introducer assembly includes at least one pawl and ratchet assembly for advancing the catheter/wire.

7. The catheter/wire introducer of claim 1, wherein the needle is removably attached to the housing.

8. The catheter/wire introducer of claim 1, wherein:
said housing is formed to define a void space for removably receiving a cartridge;
a cartridge is provided for positioning in the housing void space, said cartridge containing the catheter or wire that is inserted in the needle.

9. A catheter/wire introducer, including:
a housing shaped to be held in a single hand;
a needle that extends forward from said housing, said needle having a bore, said needle shaped to be inserted subcutaneously, the needle bore sized to receive a catheter or a wire, the catheter or wire having a conductor to which a stimulating signal can be applied;
a conductor disposed in said housing over that is connected to the needle so that a stimulating signal can be sourced to the needle;
a catheter/wire introducer assembly attached to said housing for advancing the catheter or wire through the needle bore so that a distal end of the catheter or wire can be selectively positioned forward of said needle, said introducer assembly having a manually actuatable component on said housing allowing for user control of the advancement of the catheter or wire, the manually actuatable component positioned to be actuated by the hand holding the housing;
a conductor separate from said conductor connected to said needle that is connected to the catheter or the wire over which a stimulating signal can be applied to the catheter or the wire; and
a control member attached to said housing for regulating to which component, said conductor connected to the needle or said conductor associated with the catheter or wire, a stimulating signal from a signal generator is sourced.

10. The catheter/wire introducer of claim 9, wherein said housing is formed with a window through which the catheter/wire introducer assembly manually actuatable component is accessed.

11. The catheter/wire introducer of claim 9, wherein said catheter/wire introducer assembly includes at least one pawl and ratchet assembly for advancing the catheter/wire.

12. The catheter/wire introducer of claim 9, wherein said needle is removably attached to the housing.

13. The catheter/wire introducer of claim 9, wherein:
said housing is formed to define a void space for removably receiving a cartridge;
a cartridge is provided for positioning in the housing void space, said cartridge containing the catheter or wire that is inserted in the needle.

14. A catheter/wire introducer, including:
a housing shaped to be held in a single hand;
a needle that extends forward from said housing, said needle having a bore, said needle shaped to be inserted subcutaneously, the needle bore sized to receive a catheter or a wire;
a conductor connected to the needle over which a stimulating current is applied to the needle; and
a control member attached to the housing for regulating the application of the stimulating current to the needle,
a catheter/wire introducer assembly attached to the housing for advancing a catheter or wire through the needle bore so that a distal end of the catheter or wire can be selectively positioned forward of said needle, said introducer assembly having a manually actuatable component on said housing allowing for user control of the advancement of the catheter or wire, the manually actuatable component positioned to be actuated by the hand holding the housing.

15. The catheter/wire introducer of claim 14, wherein:
said housing is formed to define a void space for removably receiving a cartridge;
a cartridge is provided for positioning in the housing void space, said cartridge containing the catheter or wire that is inserted in the needle.

16. The catheter/wire introducer of claim 14, wherein said catheter/wire introducer assembly includes a member for engaging the catheter/wire to advance the catheter/wire forward of the needle or retract the catheter/wire back into the needle.

17. The catheter/wire introducer of claim 14, wherein said housing is formed with a window through which the catheter/wire introducer assembly manually actuatable component is accessed.

18. The catheter/wire introducer of claim 14, wherein said catheter/wire introducer assembly includes at least one pawl and ratchet assembly for advancing the catheter/wire.

19. The catheter/wire introducer of claim 14, wherein said needle is removably attached to the housing.

20. The catheter/wire introducer of claim 14, further including an electrical cable that is connected to the housing for supplying the stimulating signal applied to the needle or the conductor associated with the catheter or wire.
